(19) United States
(12) Patent Application Publication
(10) Pub. No.: US 2026/0138940 A1
(43) Pub. Date: May 21, 2026

(54) PROCESS FOR METHANOL PRODUCTION

(71) Applicant: L'Air Liquide, Socièté Anonyme pour I'Etude et I'Exploitation des Procédés Georges Claude, Paris (FR)

(72) Inventors: Sebastian KOENIG, Frankfurt (DE); Vincent PENA, Frankfurt (DE)

(21) Appl. No.: 19/375,737

(22) Filed: Oct. 31, 2025

(30) Foreign Application Priority Data
Nov. 15, 2024 (EP) 24213244

(51) Int. Cl. C07C 29/151; C07C 29/80; C25B 1/04; C25B 15/023

(52) U.S. Cl. CPC C07C 29/1512; C07C 29/80; C25B 1/04; C25B 15/023

(57) ABSTRACT

The invention relates to a process for preparing methanol, in which a first reactant stream having a first reactant in the form of hydrogen and a second reactant stream having a second reactant in the form of a carbon oxide-containing gas mixture are provided; the first reactant and the second reactant are fed into a synthesis reactor arrangement; and the methanol synthesis is conducted in the synthesis reactor arrangement to form a product stream having methanol. Forecasts are created as to rates of change in inflow rate of the first and/or second reactant in order to compensate for fluctuations.

### CROSS REFERENCE TO RELATED APPLICATIONS

This application claims the benefit of priority under 35 U.S.C. § 119 (a) and (b) to European Patent Application No. 24213244.7, filed Nov. 15, 2024, the entire contents of which are incorporated herein by reference.

### BACKGROUND

### Technical Field

The present invention relates to a process and a plant for production of methanol.

### Background of the Invention

The “power-to-methanol” concept integrates the utilization of renewable energy sources by the conversion of surplus electrical energy from wind or solar power plants to chemical products. The process begins with the electrolysis of water, in which electric current is passed through an electrolyser. Water is split therein into oxygen and hydrogen. The oxidation takes place at the anode, with oxidation of water to oxygen and protons, while the protons are reduced to hydrogen gas at the cathode.

The hydrogen (H₂) produced forms the main reactant stream for the methanol synthesis. This hydrogen is combined together with carbon monoxide (CO) or carbon dioxide (CO₂) in a specific reactor equipped with a catalyst. The catalyst, often a mixture of copper, zinc oxide and aluminium oxide, promotes the chemical reaction in which hydrogen and CO/CO₂ are converted to methanol. After the synthesis, a mixture of methanol and water, called crude methanol, is condensed out of the product gas obtained and subjected to a distillation in order to remove unwanted constituents such as water and volatile and/or comparatively nonvolatile by-products. Methanol has a lower boiling temperature than water, which means that it can be purified and concentrated by distillation.

In order to ensure constant production rates in methanol synthesis and distillation, precise control of the reactant streams and the process conditions is crucial. In methanol synthesis, the supply of hydrogen and CO/CO₂ and the pressure and temperature conditions have to be regulated in a stable manner in order to ensure an efficient reaction rate and catalyst activity. Conventional plants for synthesis of methanol are designed for continuous production at constant load. Changes in loading are minimal in normal operation. Changes in load and process adjustments between defined steady states are made very slowly over hours to days, in order to protect the methanol plant and the catalyst. Rapid changes in operating conditions may impair production stability and cause unwanted by-products. For instance, a sudden change in feed rate or inflow rate of hydrogen or CO/CO₂ can greatly influence the reaction rate, which can lead to an uncontrolled increase in the temperature in the reactor, and reduces the efficiency of the reaction. This adversely affects the quality of the methanol produced. Rapid changes in inflow rates may also result in an uneven distribution of the gases, which can adversely affect reaction rate and product quality. In the distillation too, continuous operation is essential in order to assure uniform product quality. Here too, rapid fluctuations in the inflow rate of the product gas or of the crude methanol can lead to instabilities in the distillation process and impair separation efficiency. Therefore, stable feed rates and well-regulated process management are crucial in order to ensure high efficiency and product quality in both processes.

It is an object of the invention to improve the stability and efficiency of methanol production.

### SUMMARY OF THE INVENTION

The object of the invention is achieved by a process and a plant according to the independent claims. Further and preferred embodiments of the invention are disclosed in the description that follows and in the dependent claims.

The invention is described with regard to multiple aspects relating to a process and a plant. The details relating to the individual aspects are mutually complementary, and so the details of the description of the process should also be regarded as details of the description of the plant set up to perform the process, and vice versa.

The invention provides a process for preparing methanol, in which a) a first reactant stream comprising a first reactant in the form of hydrogen and a second reactant stream comprising a second reactant in the form of a carbon oxide-containing gas are provided; b) the first reactant and the second reactant are fed into a synthesis reactor arrangement; and c) the methanol synthesis is conducted in the synthesis reactor arrangement to form a product stream comprising methanol. The process further envisages the following steps: the providing of at least one first buffer for intermediate storage of at least one of the reactants upstream of the synthesis reactor arrangement; the ascertaining of the inflow rate of the at least one reactant into the synthesis reactor arrangement; and the creating of a forecast with regard to a rate of change in the inflow rate on the basis of influencing variables on which the inflow rate of the at least one reactant is dependent, wherein, when the forecast indicates a positive rate of change having a magnitude that exceeds a predefined first threshold value, preferably over a predetermined period of time, at least a portion of the stream of the at least one reactant is diverted into the first buffer; and/or, when the forecast indicates a negative rate of change having a magnitude that exceeds a predefined second threshold value, preferably over a predetermined period of time, the at least one reactant is fed from the first buffer into the synthesis reactor arrangement.

By creating a forecast as to a future rate of change in the inflow rate, predictive control of the methanol production process is possible. Predictive control of the inflow rate of the stream of the at least one reactant or reactant stream reduces the rate at which the inflow changes over time. Figuratively speaking, rather than an abrupt jump in the molar amount of the reactant in question introduced into the synthesis reactor arrangement, a gradual ramp is created over time. This makes it possible for the plant to have a gentle transition between operating states.

The inflow rate indicates how much of a particular substance flows through a system per unit time. The rate of change describes how quickly this inflow rate itself changes. When the rate of change is positive, i.e. has a positive slope, the inflow rate is increasing. If the rate of change is negative, i.e. has a negative slope, the inflow rate is decreasing. The magnitude of the rate of change indicates how quickly this change is occurring.

Threshold values are particular values that are fixed in order to monitor whether the rate of change in the inflow rate is rising or falling too quickly. The first threshold value indicates an excessively rapid increase in the inflow rate. The second threshold value indicates an excessively rapid decrease in the inflow rate. The threshold values collectively define a range within which the rate of change in the inflow rate is considered acceptable. The decision about further measures on the basis of the first or the second threshold value can be made on the basis of the slope (derivative) of the rate of change. The first threshold value is assigned a positive slope of the rate of change because it indicates an increase in the inflow rate, meaning that at least a portion of the stream of the at least one reactant is diverted into the first buffer when the forecast indicates a rate of change with a positive slope or a positive sign having a magnitude that exceeds a predefined first threshold value. The second threshold value is assigned a negative slope of the rate of change because it indicates a decrease in the inflow rate, meaning that the at least one reactant from the first buffer is fed into the synthesis reactor arrangement when the forecast indicates a rate of change with a negative slope or negative sign having a magnitude that exceeds a predefined second threshold value.

It may in principle be the case that the buffer(s) are at least partly filled in order to be prepared for changes in the inflow rate.

The first reactant and the second reactant are provided in reactant streams. The first reactant and the second reactant may be mixed in the inlet of the synthesis reactor arrangement and/or upstream of the inlet of the synthesis reactor arrangement to obtain a feed gas stream. The inflow rate may be based on the reactant per se, i.e. for example H₂, or on a reactant stream comprising the reactant. The second reactant in the form of the carbon oxide-containing gas may be a gas mixture, for example a gas including a carbon oxide-containing component. The second reactant may also be a pure carbon dioxide stream.

In the context of the process according to the invention and of the plant, the intermediate storage of the first and/or second reactant by means of first buffers includes at least the following cases:
1) A first reactant stream comprising the first reactant is provided, which may be intermediately stored in the first buffer. A second reactant stream comprising the second reactant is not connected to the first buffer.
2) A second reactant stream comprising the second reactant is provided, which may be intermediately stored in the first buffer. A first reactant stream comprising the first reactant is not connected to the first buffer.
3) A feed gas stream comprising the first reactant and the second reactant is provided, which may be intermediately stored in the first buffer.
4) A first reactant stream comprising the first reactant and a second reactant stream comprising the second reactant may be intermediately stored in separate first buffers.

The forecast or prediction of the rate of change in the inflow of one or more reactant streams into the synthesis reactor arrangement can be created with the aid of data-based prediction models. These models utilize mathematical representations of the methanol production process in order to describe the system behaviour and to carry out simulations. On the basis of historic production data, consumption patterns and other influencing variables on the reactant provision, predictions can be made with regard to the provision of H₂ and CO₂/CO. It can thus be predicted that a reduction in energy supply by X % for the H₂ electrolysis reduces hydrogen production by Y %. The prediction model is trained with training data which are assigned to the influencing variables of the reactant provision.

A control device using the prediction model monitors methanol production and calculates the probability with which a change in the inflow or inflow rate of the reactant streams will occur. In addition, the probability is calculated as to the rate of change with which the change in the inflow will occur. The model can use measurement data from a set of sensors, for example for measurement of the gas flow rates, in the methanol plant to calculate this probability. When the calculated probability exceeds a particular threshold value, the occurrence of a change in the inflow rate or the occurrence of a rate of change is considered to be forecast. If the forecast indicates a rate of change that exceeds or falls below predefined threshold values, the control device performs an assigned action in the plant.

In a further embodiment, therefore, the forecast or prediction comprises the evaluating of influencing variables, in particular performance data of operating parameters of the methanol production process on which the inflow rate of the at least one reactant is dependent or which determine the inflow rate; and the calculating of a probability of the occurrence of a rate of change, where the rate of change is considered to be forecast when the probability exceeds a predetermined threshold value.

Influencing variables, such as operating parameters of methanol production that can be used by the prediction model for creation of forecasts, include:
inflow rate of the reactant or reactant stream and influencing variables associated therewith, such as:
the providing of hydrogen, for example performance data of a water electrolyser (for example hydrogen output rate or/or the energy consumption of the electrolyser); availability of energy—in particular renewable energy—for water electrolysis, including weather conditions, electrolyte availability and volume flow rate
the providing of the carbon oxide-containing gas, for example performance data of a CO₂ separation plant or of a CO and/or synthesis gas production plant (for example CO/CO₂ output rate, pressure ratios, temperature and/or fill level)
operating parameters of the first buffers, such as fill level, temperature and/or pressure
operating parameters of the synthesis reactor arrangement, for example pressure, temperature, output rate of the product stream and/or energy consumption; inflow rate of the reactants or reactant streams
operating parameters of a distillation downstream of the synthesis

reactor arrangement, for example pressure ratios, especially pressure drop across a catalyst, temperature and/or energy consumption
operating parameters of a second buffer disposed between synthesis reactor arrangement and distillation, such as fill level, temperature and/or pressure.

The measurements of the above influencing variables may be made, for example, by means of sensors distributed within a plant for producing methanol. The enumeration of the above influencing variables is not exhaustive. Further and/or other parameters that can be used for a forecast of the rate of change can be derived from the computational modelling of the underlying system for methanol production.

In order to set the rate of change in the inflow rate of a gas stream into the synthesis reactor arrangement to a predefined value or predefined range, a closed-loop control system may be provided, for example a closed-loop control system implemented as part of a control device in a data processing unit with a hardware processor. The closed-loop control system is preferably set up to perform the following steps:
Measuring the actual value of the inflow rate of the at least one reactant or reactant stream.
Creating a target value for feeding of the reactant or reactant stream into the synthesis reactor arrangement. The target value can be determined on the basis of the construction of the synthesis reactor arrangement, for example by utilization of a model that takes account of the operating conditions and the power requirements of the plant.
Comparing the actual value with the predefined target value.
Determining and adjusting a control variable that influences the inflow rate on the basis of the variance found between target value and actual value.

Useful control variables include:
the diverting of the gas streams, for example the at least one reactant, into a first buffer tank or by the discharging of the at least one reactant from the first buffer tank, for example by means of valves and pumps, electrical actuating elements, electrical circuits, etc., the directing of heat flows and electrical energy

The closed-loop control system operates continuously in a feedback loop by measuring the actual value of the gas flow, comparing it with the target value of the gas flow rate and correspondingly adjusting the control variables. In this way, the inflow rate of the reactant stream in question is actively regulated and the rate of change is controlled to the desired range in order to ensure a stable and reliable process regime.

The intermediate storage of at least one of the reactants in the first buffer is preferably such that the first reactant is stored in the form of gaseous hydrogen. The second reactant in the form of the carbon oxide-containing gas is preferably stored in liquid form.

In a further embodiment of the invention, the first and the second reactant are provided in separate reactant streams, where a separate buffer is provided for at least one of the reactant streams.

In a further embodiment of the invention, the two reactant streams are intermediately stored in separate first buffer stores.

In a further embodiment of the invention, the product stream produced in the synthesis reactor arrangement is passed into a distillation. It is also envisaged that a second buffer is provided for intermediate storage of the product stream and that the inflow rate of the product stream into the distillation is ascertained. It is further envisaged that the product stream is at least partly diverted into the second buffer or a product stream is fed from the second buffer into the distillation in order to compensate for changes in the inflow rate of the product stream from the synthesis reactor arrangement. The further buffer enables more stable supply of the product stream to the distillation. Since the inflow rate from the synthesis reactor arrangement can vary, the buffer serves as an intermediate storage device that absorbs fluctuations in the product flow rate. This means that the distillation is supplied with a more uniform feed, which increases the efficiency of the separation processes.

In a further embodiment of the invention, a forecast is created with regard to a rate of change in the inflow rate of the product stream into the distillation on the basis of influencing variables on which the inflow rate of the product stream is dependent. If the forecast indicates or predicts a positive rate of change having a magnitude that exceeds a predefined third threshold value, at least a portion of the product stream is diverted into the second buffer; and/or, when the forecast indicates or predicts a negative rate of change having a magnitude that exceeds a predefined fourth threshold value, a product stream is fed from the buffer into the distillation, in order to adjust the rate of change in the inflow rate of the product stream into the distillation to a predefined value or predefined range. A forecast as to the product flow rate or the product stream inflow rate can be created on the same basis as elucidated in relation to the reactant stream(s). Influencing variables and operating parameters which can be used to create the forecast include the variables already elucidated in relation to the reactant streams.

In a further embodiment of the invention, the second reactant is carbon dioxide and/or carbon monoxide and/or a synthesis gas mixture comprising at least hydrogen and carbon monoxide.

The hydrogen is preferably provided by water electrolysis, preferably at least partly with renewable energy. Preference is given to using an electrolyser fluidically connected to the synthesis reactor arrangement, meaning that the electrolyser is incorporated into the plant having the synthesis reactor arrangement.

In a further embodiment of the invention, the creating of a forecast as to the inflow rate of the first reactant includes a prediction with regard to the provision of energy for the water electrolysis. This may in particular be based on technical or commercial influencing variables which influence or determine the provision of energy for the water electrolysis. Future changes in the availability of energy for the electrolysis can be estimated or determined in various ways. One possibility is the analysis of historic data in order to understand past energy consumption patterns and trends for similar processes or plants. In addition, market analyses play a role in that they take account of factors such as energy costs and the availability of energy sources that can affect future availability. Finally, simulations enable the modelling of different scenarios for the provision of energy, for example by simulating the energy expenditure and energy availability of an entire plant, for example of a site, and evaluating it under different operating conditions. These principles can be used to set up a prediction as to the provision of energy for the electrolysis, which in turn enables prediction of the inflow rate of the first reactant. This is advantageous in particular with regard to the renewable energies, since their availability is often subject to fluctuations.

In a further embodiment of the invention, the first reactant is provided by water electrolysis and a forecast is created with regard to the provision of energy for the water electrolysis on the basis of parameters that influence or determine the provision of energy for the water electrolysis. Also envisaged is a ramp-up operation in which a forecast increase in an energy provision for the water electrolysis leads to an increase in the inflow rate of the first reactant, wherein the first reactant is at least partly intermediately stored in the first buffer in such a way that the rate of change in the inflow rate of the first reactant fed into the synthesis reactor arrangement is limited to a predefined rate. It is thus possible to react optimally to a forecast increase in hydrogen production as a result of increasing energy availability. In particular, the supply of hydrogen can be limited to an acceptable or optimal rate for methanol synthesis. In a further embodiment of the invention, alternatively or additionally, a ramp-down operation is envisaged, in which a forecast decrease in an energy supply for the water electrolysis leads to a reduction in the inflow rate of the first reactant, wherein the first reactant is fed from the first buffer into the synthesis reactor arrangement in such a way that the rate of change in the inflow rate of the first reactant fed into the synthesis reactor arrangement is limited to a predetermined rate. It is thus possible to react optimally to a forecast reduction in hydrogen production as a result of decreasing energy availability. While methanol production in the methanol synthesis is decreasing, the second buffer fulfils a buffer function in order to limit the methanol feed to an effective amount for the methanol distillation.

In a further embodiment of the invention, heat is withdrawn from the synthesis reactor arrangement and/or the distillation and intermediately stored and/or generated by energy supply, wherein the process further comprises standby operation, wherein standby operation is commenced when the forecast inflow rate of at least one of the first and second reactants into the synthesis reactor arrangement, preferably over a predetermined period of time, falls below a predetermined threshold value, wherein pressure and temperature conditions in the synthesis reactor arrangement are regulated to predetermined values, wherein the heat generated by energy supply is conducted into the synthesis reactor arrangement and/or the distillation. Pressure and temperature conditions are maintained by introducing heat into the synthesis reactor arrangement. As a result, rapid startup of regular operation is possible after standby operation has been cancelled. Preferably, in standby operation, the inflows of the first reactant and/or second reactant into the synthesis reactor arrangement are stopped. Standby operation is energy-efficient and enables a flexible operating strategy.

In a further embodiment of the invention, standby operation is cancelled again when a forecast inflow rate of at least one of the reactant streams reaches a predetermined threshold value, preferably over a predetermined period of time, in which case the introduction of the heat into the synthesis reactor arrangement and/or the distillation can be stopped. In addition, the feeding of first and/or second reactant is resumed.

A further aspect of the invention relates to a plant for methanol production, comprising a synthesis reactor arrangement, a distillation; one or more sources for a first reactant gas and a second reactant gas, wherein the one or more sources are connected to the synthesis reactor arrangement via a common first conduit or via separate first conduits, wherein at least one of the conduits is connected to a first buffer; and a second conduit (crude methanol conduit) which connects the synthesis reactor arrangement to the distillation, wherein the second conduit is connected to a second buffer; and a control device set up to operate the plant by one of the processes described herein.

The control device can run as software on a data processing device with a hardware processor. This may have the control systems and forecast functions described herein in the form of software. The software can receive and evaluate signals from sensors provided in the plant, and also further data, for example the energy availability from the power grid, which are processed as operating parameters and influencing variables for the creation of the forecasts and the open-loop and closed-loop control of the plant. In addition, the control device can be operatively connected to actuating elements or control elements of the plant in order to control the plant. The actuating elements may include valves, pumps, electrical switches, energy supply and further control elements that can be used for open-loop and closed-loop control, in particular of the gas flow rates, heat feed, etc., to a plant by means of the control device.

### BRIEF DESCRIPTION OF THE FIGURE

For a further understanding of the nature and objects for the present invention, reference should be made to the following detailed description, taken in conjunction with the accompanying drawing, in which like elements are given the same or analogous reference numbers and wherein:

FIG. 1 shows a schematic diagram of a first embodiment of a plant according to the invention for methanol production.

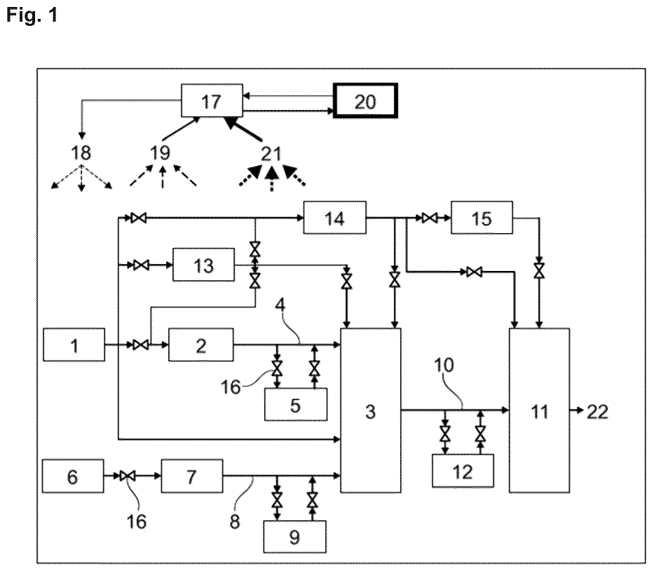

### DETAILED DESCRIPTION OF PREFERRED EMBODIMENTS

The plant comprises an energy supply 1, an electrolyser 2 for hydrogen electrolysis, i.e. for generating a first reactant in the form of hydrogen (H₂). The electrolyser 2 is supplied with power via the energy supply 1, which indicates the connection between the energy supply 1 and the electrolyser 2.

The electrolyser 2 is fluidically connected to a synthesis reactor arrangement 3 for methanol synthesis via a first conduit 4. The first conduit 4 is connected to a first buffer 5.

A CO₂ source 6 is fluidically connected to a CO₂ separation unit 7, which in turn is fluidically connected to the synthesis reactor arrangement 3 via a second conduit 8. The second conduit 8 is connected to a further first buffer 8 for CO₂.

The synthesis reactor arrangement 3 is connected to a methanol distillation 11 via a crude methanol conduit 10. The crude methanol conduit 10 is connected to a second buffer 12.

The plant further comprises an energy storage means 13 for storing electrical energy, a power-to-heat converter 14 and a storage means for thermal energy 15. The energy storage means 13 and the power-to-heat converter 14 are electrically connected to the energy supply 1. The storage means for thermal energy 15 is thermally connected to the power-to-heat converter 14.

The connections shown between the above-described components are functionally active connections. These are in particular electrical wires between electrical components; these are in particular gas or fluid conduits between components that are fluidically connected. The symbols 16 shown in the connections represent actuating elements, such as switching elements between electrical components, or valves between components that are fluidically connected. By means of the actuating elements 16 between the first buffer 5 and the first conduit 4, it is possible, for example, to control the inflow of hydrogen from the electrolyser 2 into the first buffer 5 and from the latter into the first conduit 4.

A control device 17 runs on a data processing device (not shown). The latter can send control commands 18, for example wirelessly, to the actuating elements 16. Moreover, the control device 17 receives, in real time, performance data 18 of operating parameters, for example the inflow rate of hydrogen from the electrolyser 2 into the synthesis reactor arrangement 3, via sensors (not shown) distributed within the plant, in particular in the components shown (electrolyser, synthesis reactor arrangement, distillation, etc.) and the conduits.

The control device 17 exchanges data with a simulation/prediction model 20 that likewise runs on the data processing device. The simulation/prediction model 20 is a mathematical representation of the plant and receives real-time data 18 from the plant and uses these data to model the behaviour of the plant in real time and to predict changes in operating states by means of prediction models. The simulation/prediction model 20 can predict, based on the current operating conditions and further parameters, the availability of electrical energy in a next period of time and how much hydrogen will be produced in the next period of time. These forecast data 21 are passed to the control device 17.

In the regular operating mode, the energy supply 1 provides energy for the electrolyser 2. This produces a first reactant in the form of hydrogen. This enters the synthesis reactor arrangement 3 as first reactant stream via the first conduit 4. In addition, a second reactant stream comprising a second reactant in the form of CO₂ is fed into the synthesis reactor arrangement 3 via the second conduit 8. The methanol synthesis takes place therein according to the reaction CO₂+3H₂→CH₃OH+H₂O. This gives rise to a product stream comprising crude methanol, which is fed into the methanol distillation 11 via the crude methanol conduit 10. After the distillation, pure methanol 22 is discharged.

Three operating modes are presented below by way of example.

### Ramp-Up Operation

In a ramp-up operation, an excessively rapid increase in the inflow rate of CO₂ and/or H₂ into the synthesis reactor arrangement 3 is compensated for. By way of example, a ramp-up operation is described, in which an increase in the energy supply for the electrolyser 2 is predicted. The simulation model 20 receives real-time data relating to operating parameters from the components of the plant. On the basis of model calculations and historic data inter alia, the control device 17 calculates, with the aid of the simulation/prediction model 20, a forecast as to the energy provided for a future period of time. A forecast increase in energy supply would lead to a major increase in hydrogen production by the electrolyser 2 and in hydrogen inflow into the synthesis reactor arrangement 3. The control device 17, in association with the simulation/prediction model 20, then calculates the speed with which the inflow rate of hydrogen will change. This speed is expressed by the rate of change in the inflow rate.

The control device 17 compares the slope (positive or negative) of the forecast rate of change in the inflow rate of the hydrogen and its magnitude with stored rate-of-change values. If it is determined that the magnitude of a forecast rate of change with a positive sign exceeds a predefined threshold value for a particular period of time, the control device 17 commences compensation measures even before the occurrence of the energy rise and diverts a portion of the first reactant stream from the electrolyser 2 in the first conduit 4 into the first buffer 5 by actuating the corresponding valves between first conduit 4 and first buffer 5. This lowers the rate of change in the inflow rate of the reactant stream. The first buffer 5 serves here as buffer. The diverting of the hydrogen additionally limits the inflow rate of the hydrogen to a maximum inflow rate effective for methanol synthesis.

As the inflow rate of hydrogen rises, there is also a rise in the amount of product gas and hence of condensed crude methanol which is discharged from the synthesis reactor arrangement 3 and passed to the distillation 11. In order to limit the inflow rate of crude methanol into the distillation 11, but also the rate of change in the inflow rate of crude methanol, to a maximum rate effective for the distillation, the control device 17 actuates valves in the crude methanol conduit 10 in order to divert a portion of the crude methanol into the second buffer 12.

When methanol production in the synthesis reactor arrangement 3 reaches the processing capacity of the distillation 11, the excess of crude methanol is accommodated in the second buffer 12.

### Ramp-Down Operation

In ramp-down operation, an excessively rapid drop in the inflow rate of CO₂ and/or H₂ into the synthesis reactor arrangement 3 is compensated for. A rapid drop in the inflow rate may be caused, for example, by a major drop in energy supply. If the control device 17 predicts a drop in energy supply and an associated drop in hydrogen production and the inflow rate of hydrogen, the likewise forecast rate of change in the inflow rate is compared with a predefined threshold value. If the magnitude of a forecast rate of change in the inflow rate with a negative sign is above a predefined threshold value for a particular period of time, via the actuation of valves between the first buffer 5 and the first conduit 4, hydrogen intermediately stored in the first buffer 5 is fed into the first conduit 4 and the synthesis reactor arrangement 3. The first buffer 5 compensates for the rate of reduction of the hydrogen supply into the synthesis reactor arrangement 3 to a degree effective for methanol synthesis. In addition, the first buffer 5 is used to keep the inflow rate of the hydrogen at an inflow rate effective for methanol synthesis if hydrogen production should continue to drop.

As the inflow rate of hydrogen drops, there is also a drop in the amount of crude methanol which is discharged from the synthesis reactor arrangement 3 and passed to the distillation 11. In order to limit the inflow rate of crude methanol into the distillation, but also the rate of change in the inflow rate of crude methanol, to rates effective for the distillation, the control device 17 actuates valves in the crude methanol conduit 10 in order to feed crude methanol from the second buffer 12 into the distillation 11.

If methanol production in the synthesis reactor arrangement 3 is below a minimum processing capacity limit of the distillation 11, the operation of the distillation is first maintained by feeding in crude methanol from the second buffer 12. If no further crude methanol can be provided, the distillation 11 can be put into a reflux mode in which distillation products are circulated internally.

### Standby Operation

The control device 17 is also designed for standby operation. If the control device 17 forecasts that the hydrogen production is inadequate or cannot take place for a prolonged period of time, the plant can be put into standby mode. In this mode, pressure and temperature conditions in the synthesis reactor arrangement 3 are set to a defined level in order to assure rapid startup of the synthesis reactor arrangement 3 after standby operation has ended. For this purpose, heat is introduced into the synthesis reactor arrangement 3 and the distillation via the power-to-heat converter 14 and the storage means for thermal energy 15.

### List of Reference Numerals

1 energy supply
2 electrolyser
3 synthesis reactor arrangement
4 first conduit
5 first buffer
6 CO2 source
7 CO2 separation unit
8 second conduit
9 further first buffer
10 crude methanol conduit
11 methanol distillation
12 second buffer
13 energy storage means
14 power-to-heat converter
15 storage means for thermal energy
16 actuating element
17 control device
18 control command
19 real-time data
20 simulation/prediction model
21 forecast data
22 pure methanol

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A process for producing methanol, comprising:
a) a first reactant stream comprising a first reactant in the form of hydrogen and a second reactant stream comprising a second reactant in the form of a carbon oxide-containing gas are provided;
b) the first reactant and the second reactant are fed into a synthesis reactor arrangement; and
c) the methanol synthesis is conducted in the synthesis reactor arrangement to form a product stream comprising methanol;
wherein the process further comprises:
providing at least one first buffer for intermediate storage of at least one of the reactants upstream of the synthesis reactor arrangement;
determining the inflow rate of the at least one reactant into the synthesis reactor arrangement; and the
creating of a forecast with regard to a rate of change in the inflow rate on the basis of influencing variables on which the inflow rate of the at least one reactant is dependent;
wherein
when the forecast indicates a positive rate of change having a magnitude that exceeds a predefined first threshold value, at least a portion of the stream of the at least one reactant is diverted into the first buffer; and/or
when the forecast indicates a negative rate of change having a magnitude that exceeds a predefined second threshold value, the at least one reactant is fed from the first buffer into the synthesis reactor arrangement,
thereby adjusting the rate of change to a predefined value or predefined range.

2. The process according to claim 1, wherein the first and the second reactants are provided in separate reactant streams, wherein a separate buffer is provided at least for one of the reactant streams.

3. The process according to claim 1, wherein the first and the second reactant streams are stored intermediately in separate first buffers.

4. The process according to claim 1, further comprising:
d) the feeding of the product stream produced in the synthesis reactor arrangement into a distillation;
providing of a second buffer for intermediate storage of the product stream; and
determining the inflow rate of the product stream into the distillation;
wherein the product stream is at least partly diverted into the second buffer or a product stream is fed from the second buffer into the distillation in order to compensate for changes in the inflow rate of the product stream from the synthesis reactor arrangement.

5. The process according to claim 4, further comprising
creating a forecast with regard to a rate of change in the product flow rate into the distillation on the basis of influencing variables on which the inflow rate of the product stream is dependent,
wherein
when the forecast indicates a positive rate of change having a magnitude that exceeds a predefined third threshold value, at least a portion of the product stream is diverted into the second buffer; and/or
when the forecast indicates a negative rate of change having a magnitude that exceeds a predefined fourth threshold value, a product stream is fed from the buffer into the distillation,
thereby adjusting the rate of change in the inflow rate of the product stream into the distillation to a predefined value or predefined range.

6. The process according to claim 1, wherein the second reactant is carbon dioxide and/or carbon monoxide and/or a synthesis gas mixture comprising at least hydrogen and carbon monoxide.

7. The process according to claim 1, wherein the hydrogen is provided by water electrolysis.

8. The process according to claim 7, further comprising creating a forecast with respect to the provision of energy for the water electrolysis on the basis of parameters that influence the provision of energy for the water electrolysis.

9. The process according to claim 8, wherein the process further comprises:
a) a ramp-up operation in which a forecast increase in an energy provision for the water electrolysis leads to an increase in the inflow rate of the first reactant, wherein the first reactant is at least partly intermediately stored in the first buffer in such a way that the rate of change in the inflow rate of the first reactant fed into the synthesis reactor arrangement is limited to a predefined rate; and/or
b) a ramp-down operation in which a forecast decrease in an energy supply for the water electrolysis leads to a reduction in the inflow rate of the first reactant, wherein the first reactant is fed from the first buffer into the synthesis reactor arrangement in such a way that the rate of change in the inflow rate of the first reactant fed into the synthesis reactor arrangement is limited to a predetermined rate.

10. The process according to claim 1, wherein heat is withdrawn from the synthesis reactor arrangement and/or the distillation and intermediately stored and/or generated by energy supply,
wherein the process further comprises standby operation,
wherein standby operation is commenced when the forecast inflow rate of at least one of the first and second reactants into the synthesis reactor arrangement falls below a predetermined threshold value,
wherein pressure and temperature conditions in the synthesis reactor arrangement and/or the distillation are regulated to predetermined values, wherein the heat generated by energy supply is conducted into the synthesis reactor arrangement and/or the distillation.

11. The process according to claim 10, wherein standby operation is cancelled again when a forecast inflow rate of at least one of the reactant streams reaches a predetermined threshold value, wherein the introduction of the heat is preferably stopped.

12. The process according to claim 1, wherein
the first reactant is intermediately stored in the first buffer,
the second reactant is intermediately stored in the first buffer;
the first and second reactants are intermediately stored together in the first buffer; or
the first reactant and the second reactant are intermediately stored in separate first buffers.

13. The process according to claim 1, wherein the creating of forecasts comprises the evaluating of influencing variables, ; and the calculating of a probability of the occurrence of a rate of change, wherein, when the probability exceeds a predetermined threshold value, the rate of change is considered to be forecast.

14. A plant for methanol production, comprising:
a synthesis reactor arrangement,
a distillation;
one or more sources for a first reactant gas and a second reactant gas, wherein the one or more sources are connected to the synthesis reactor arrangement via a common first conduit or via separate first conduits,
wherein at least one of the conduits is connected to a first buffer; and
a second conduit which connects the synthesis reactor arrangement to the distillation, wherein the second conduit is connected to a second buffer;
a control device set up to operate the arrangement by a process according to claim 1.
